(19) United States
(12) Patent Application Publication
(10) Pub. No.: US 2004/0176829 A1
(43) Pub. Date: Sep. 9, 2004

(54) Electrode device for myocardial and the like

(75) Inventor: Masayoshi Yokoyama, Tokyo (JP)

Correspondence Address: JORDAN AND HAMBURG LLP, 122 EAST 42ND STREET, SUITE 4000, NEW YORK, NY, 10168, US

(73) Assignee: Japan General Medical Institute Co., Ltd., Suginami-ku (JP)

(21) Appl. No.: 10/378,700

(22) Filed: Mar. 4, 2003

(51) Int. Cl. A61N 1/05

(52) U.S. Cl. 607/129; 607/130

(57) ABSTRACT

An electrode device for myocardial and the like comprises an electrode device body with elasticity; the electrode parts provided in a tip part of the electrode device body or a portion adjacent the tip part of the electrode device body, having a prevention part for missing the electrode parts; and the grip parts provided so as to nip the electrode device body so that it can avoid fear of infection disease and heart failure and connect the electrode device from an epimyocardium side

### BACKGROUND OF THE INVENTION

This invention relates to an electrode device for myocardial and the like.

Conventionally, the lead of the pacemaker is inserted in right ventricular cardiac apex (or right atrial appendage of the ventriculus dexter) from the inside of a vein. In this operation, the structure of cardiac apex is suitable for taking in a tip portion of the lead so that the lead cannot escape easily, and the operation is also easy so that about 95% or more of the pacemaker operation is performed by the above-mentioned method because the operation finishes within 30 minutes. Moreover, the method of thrusting and fixing the electrode formed in the shape of a coil into myoepicardial mantle is performed.

In the conventional method, since blood is contact with the lead, it will generate heat when the cause is a bacterial infection, and it causes life-threatening blood poisoning and a patient may become a serious condition. Moreover, since a lead must pass a tricuspid valve in order to insert it in the right ventricle, some patients fall tricuspid insufficiency (TI), heart failure and the like. Furthermore, the vein that inserted the lead closes. Moreover, when the coiled electrode is used, the depth in which the tip part of electrode is inserted is restricted naturally, it rotates in the direction from which electrode separates by a twist or distortion and floats, then an insulated state is caused between epimyocardium and myocardium.

### SUMMARY OF THE INVENTION

In light of the forgoing, it is an object of the invention to provide an electrode device for myocardial and the like which can avoid fear of infection disease and heart failure and connect the electrode device from an epimyocardium side.

Novel features which are believed to be characteristic of the invention, both as to its organization and method of operation, together with further objects and advantages thereof, are described below with reference to the accompanying drawings in which preferred embodiments of the invention are illustrated as an example.

It is to be expressly understood, however, that the drawings are for the purpose of illustration and description only, and are not intended as a definition of the limits of the invention.

### BRIEF DESCRIPTION OF THE DRAWINGS

FIG. 1 is a perspective view showing a first embodiment of the present invention;

FIG. 2 is a cross sectional view taken along a line 2-2 of FIG. 1;

FIG. 3 is an explanation view of the way in which an electrode device is attached;

FIG. 4 is an explanation view of the way in which an electrode device is attached;

FIG. 5 is an explanation view in use

FIG. 6 is a perspective view showing a second embodiment of the present invention;

FIG. 7 is a perspective view showing a third embodiment of the present invention;

FIG. 8 is a cross sectional view taken along a line 8-8 of FIG. 7;

FIG. 9 is a perspective view showing a fourth embodiment of the present invention;

FIG. 10 is a perspective view showing a fifth embodiment of the present invention;

FIG. 11 is a perspective view showing a sixth embodiment of the present invention;

FIG. 12 is a perspective view showing a seventh embodiment of the present invention;

FIG. 13 is a perspective view showing an eighth embodiment of the present invention; and

FIG. 14 is a perspective view showing a ninth embodiment of the present invention.

### DETAILED DESCRIPTION OF THE PREFERRED EMBODIMENTS

Preferred embodiments of the present invention are described in more detail below with reference to the accompanying drawings.

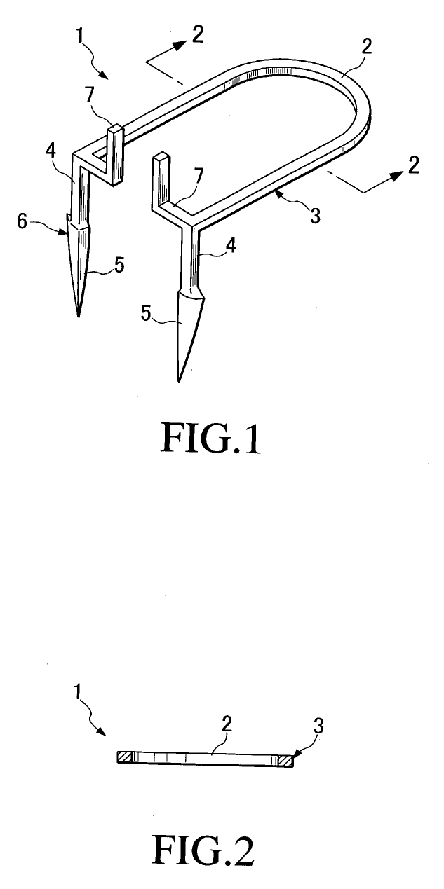

An understanding of the present invention may be best gained by reference to FIGS. 1-5. FIGS. 1-5 illustrate an electrode device for myocardial and the like in accordance with a first embodiment of the present invention.

The reference number 1 shows a myocardial electrode device wherein is composed of an electrode device body 3; electrode parts 6; and grip parts 7. The electrode device body 3 is formed from metal material which has the elasticity and does not have toxicity in living bodies, such as, for example, acid-proof stainless steel, stainless steel, platinum, iridium and so on, forming in the shape of a U-letter and plate in the cross sectional view, and having a spring part 2. The electrode part 6 further includes a electrode part body 4 provided at a tip end portion or a portion adjacent the tip end portion of the electrode device body 3, the tip end portion in this embodiment, forming for the same material of the electrode device body; and a prevention part 5 for missing from the epimyocardium, forming so as to project to the moving direction of the electrode part body 4 and forming in the shape of a needle, such as, for example fishing hook. The grip part 7 is provided at the tip portion of the electrode device body 3 so as to move to the inner direction of the electrode device body 3, forming the same material as the electrode device body 3.

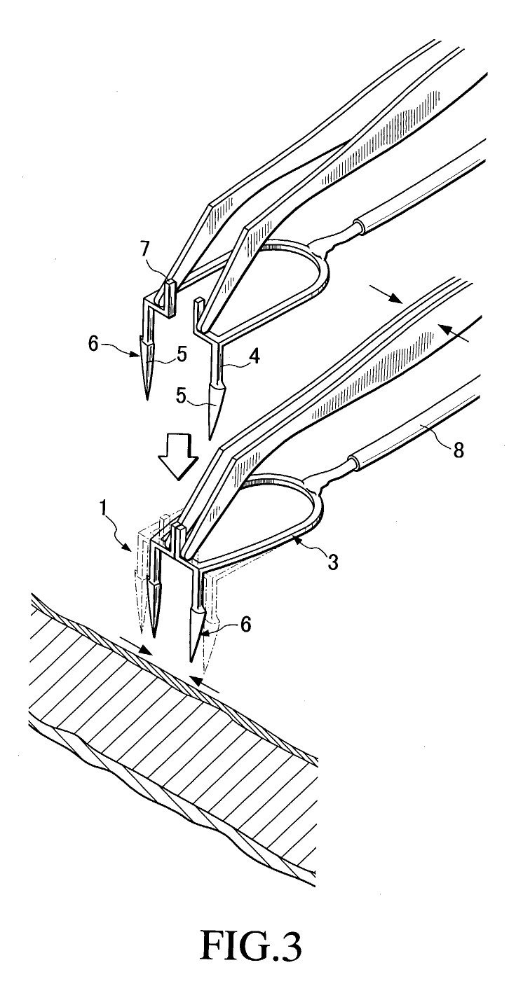

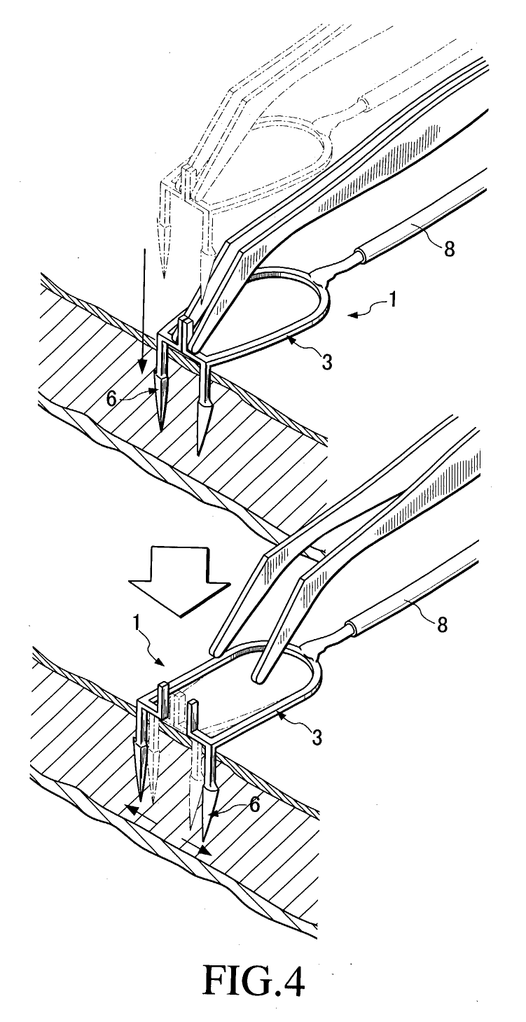

The length of the electrode part 6 is about 2-15 mm, preferably about 5 mm in length in this embodiment. Then its length may change based on the fat volume on a surface of the patient's heart and portion to the heart to insert. In addition, the width between the electrode parts 6 is about 2-12 mm, preferably about 5-10 mm in width in this embodiment, and the width can be adjusted by the spring power of the spring part 2. In the myocardial electrode device 1 of the above-mentioned composition, as shown in FIGS. 3 and 4, the lead 8 is connected to the electrode device body 3, the grip part 7 is nipped by the nip part of the electrode-insert means, such as, for example, tweezers, pliers and so on. Then, the myocardial electrode device 1 is inserted through the small hole (port) made to chest wall, and the electrode part 6 is inserted in the predetermined part of the epimyocardium. After the nip part is opened wide and the electrode-insert means is pulled out, the electrode parts 6 moves to the expanded (outer) direction in the action of spring power.

Therefore, each electrode part 6 moves to the different myocardial tissue from the inserted portion so that the damage of the part the electrode part 6 stops is just a slight injury rather that that of the part the electrode part 6 is inserted, and maintenance power of myocardial is high and the it can prevent to miss the electrode parts 6. Moreover, since the prevention part 5 does not injure a myocardial tissue, the support power is maintainable.

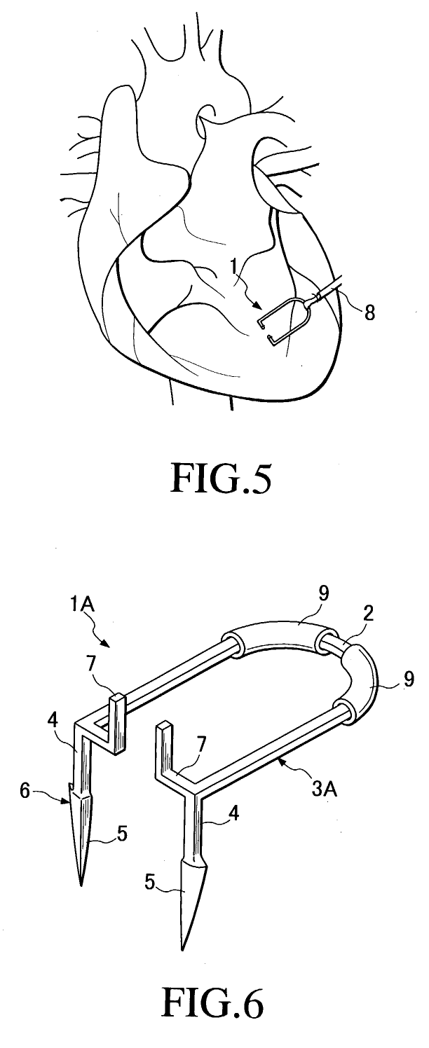

In addition, as shown in FIG. 5, the myocardial electrode device 1 of the present invention can be used as the electrode of the pacemaker, defibrillator and so on. Therefore, the same polarity, i.e., the anode and anode, and the negative and negative are sufficient, for example, when it is used for the pacemaker, the heart side is a minus pole and the electrode parts side becomes a plus pole.

Moreover, it can be used for multi-site pacing device provided with a plurality of the electrode.

Moreover, although the electrode device body 3, the electrode parts 6 and the grip parts 7 are formed with the same material, they may be formed a different material respectively, for example, the spring part 2 of the electrode device body 3 may be formed with another material. At least the electrode parts 6 are formed with a conductive material.

Other embodiments of the present invention will now be described with reference to FIGS. 6-14. In FIGS. 6-14, the same components as in the first embodiment described above with reference to FIGS. 6-14 are designated by the same reference numerals and therefore will not be further explained in great detail.

A second embodiment of the present invention is shown in FIG. 6. It is distinguished from the first embodiment in that an insulated part 9 is formed in the electrode device body 3 or a part between the electrode device body 3 and electrode parts 6, the spring part of the electrode device body 3 in this embodiment, forming with insulated material, such as, for example, rubber and synthetic resin. An electrode device 1A with the electrode device body 3A according to the second embodiment has similar advantages to that according to the first embodiment.

In this case, one electrode part 6 is the anode pole and another is a cathode pole. Therefore, when it is used for the pacemaker, the negative pole and the plus pole will also be attached to the heart side.

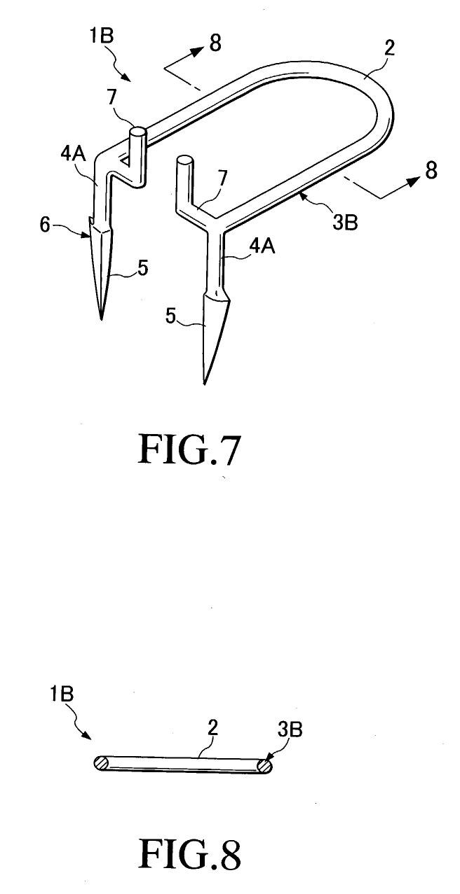

A third embodiment of the present invention is shown in FIGS. 7 and 8. It is distinguished from the first embodiment in that an electrode device body 3B and electrode part body 4A are formed in the shape of a circle in a cross-sectional. An electrode device 1B according to the third embodiment has similar advantages to that according to the first embodiment.

In addition, the grip parts 7 may be similarly formed.

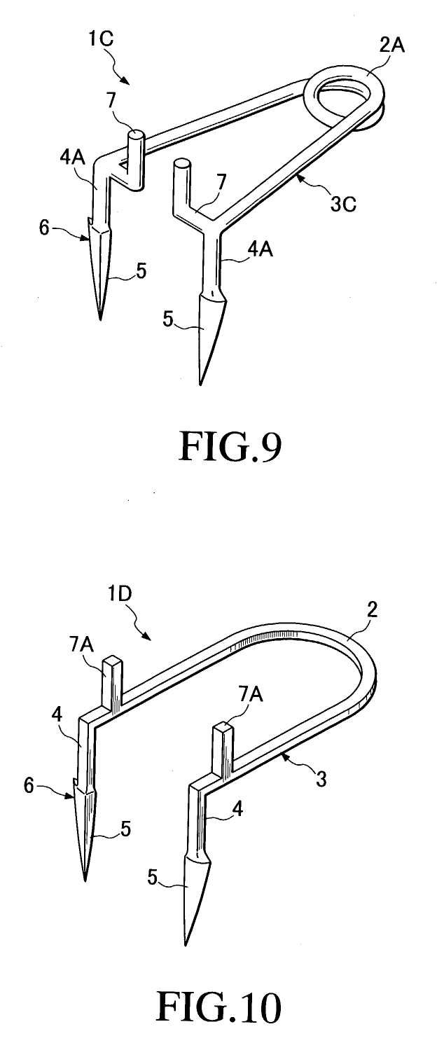

A fourth embodiment of the present invention is shown in FIG. 9. It is distinguished from the third embodiment in that a spring part 2A is formed in the shape of a spring. An electrode device 1C with the electrode device body 3C according to the fourth embodiment has similar advantages to that according to the third embodiment.

A fifth embodiment of the present invention is shown in FIG. 10. It is distinguished from the first embodiment in that each grip part 7A is formed at an upper surface of the electrode device body 3 so as to project to the upward direction. An electrode device 1D according to the fifth embodiment has similar advantages to that according to the first embodiment.

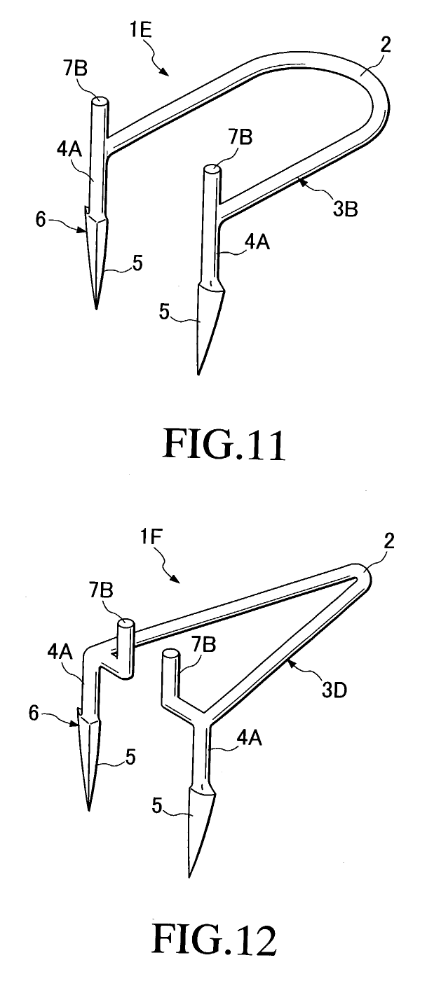

A sixth embodiment of the present invention is shown in FIG. 11. It is distinguished from the third embodiment in that each grip parts 7B is formed on the vertical direction of the electrode parts 6 so to project to the upward direction. An electrode device 1E according to the sixth embodiment has similar advantages to that according to the third embodiment.

A seventh embodiment of the present invention is shown in FIG. 12. It is distinguished from the third embodiment in that electrode device body 3D is formed in the shape of V-letter. An electrode device 1F according to the seventh embodiment has similar advantages to that according to the third embodiment.

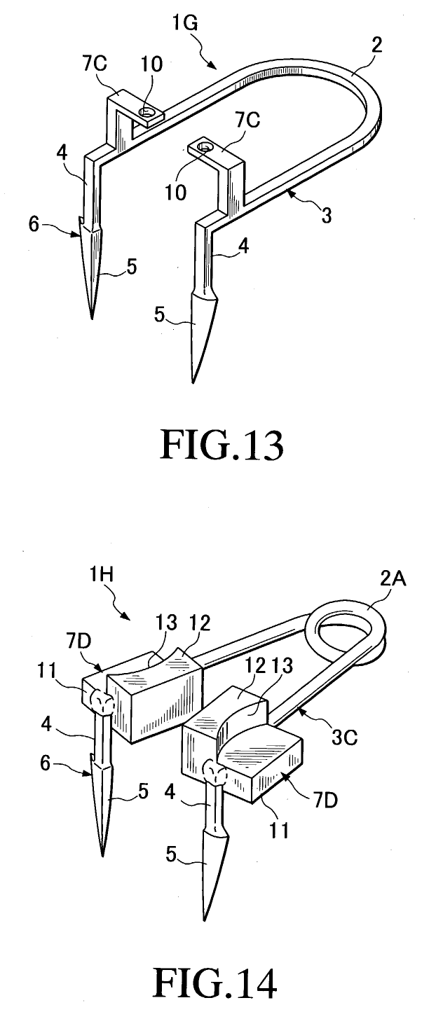

An eighth embodiment of the present invention is shown in FIG. 13. It is distinguished from the first embodiment in that each grip parts 7C is formed in the shape of a reversed L-letter, having a insertion hole 10 which is provided at a tip portion thereof. An electrode device 1G according to the eighth embodiment has similar advantages to that according to the first embodiment.

A ninth embodiment of the present invention is shown in FIG. 14. It is distinguished from the fourth embodiment in that each grip parts 7D is further included a first grip base 11 and a second grip base 12 provided on the first grip base 11, having a concave part 13 which is formed at a side portion thereof. An electrode device 1H according to the ninth embodiment has similar advantages to that according to the fourth embodiment.

As set forth above, the advantages of the invention are as follows:

(1) An electrode device for myocardial and the like comprises an electrode device body with elasticity; electrode parts provided in a tip part of the electrode device body or a portion adjacent the tip part of the electrode device body, having a prevention part for missing the electrode parts; and grip parts provided so as to nip the electrode device body so that it can connect to the myocardial from an epimyocardium side.

Therefore, it can avoid fear of infection or heart failure easily.

(2) As discussed above, since the electrode parts move to the different part of the myocardial tissue from the inserted part and it is certainly fixed to the myocardial by the prevention part, it can prevent to fall the electrode out efficiently without injuring the myocardial tissue.

Therefore, a patient can be watched in comfort during the operation or after the operation.

(3) As discussed above, since it has a simple structure, it can manufacture easily.

Therefore, it can reduce its cost.

What is claimed is:

1. An electrode device for myocardial and the like comprising:
an electrode device body with elasticity;
electrode parts provided in a tip part of the electrode device body or a portion adjacent the tip part of the electrode device body, having a prevention part for missing the electrode parts; and
grip parts provided so as to nip the electrode device body.

2. An electrode device for myocardial and the like according to claim 1, further includes an insulated part provided in the electrode device body or a portion between the electrode device body and the electrode parts.

3. An electrode device for myocardial and the like according to claim 1, wherein the electrode device body further includes a spring part.

4. An electrode device for myocardial and the like according to claim 1, wherein each electrode part is provided so as to project to the vertical direction against the electrode device body.
